(19) United States
(12) Patent Application Publication
(10) Pub. No.: US 2005/0220860 A1
(43) Pub. Date: Oct. 6, 2005

(54) Stabilized pure vitamin-C in powder to liquid form using multi-encapsulation method

(76) Inventor: Dong Yong Kim, Los Angeles, CA (US)

Correspondence Address: Eugene Oak, Ph.D.,J.D.;Patent Attorney, 610 S. Van Ness Ave., Los Angeles, CA, 90005, US

(21) Appl. No.: 11/139,762

(22) Filed: May 27, 2005

Related U.S. Application Data
(63) Continuation-in-part of application No. 10/122,360, filed on Apr. 15, 2002.

(51) Int. Cl. A61K 9/127

(52) U.S. Cl. 424/450

(57) ABSTRACT

Current application relates to a cosmetic powder that contains stabilized pure vitamin C (ascorbic acid) by utilizing ‘multi-encapsulation method.’ Powders of pure vitamin C are well blended with mixtures of glycerin and lecithin to form a liposome of liquid emulsion state. The emulsion, which is comprised of vitamin C, glycerin and lecithin, is encapsulated with CAB-O-SIL® TS-530, which is micro particle of silica treated with hexamethyldisilazane. On the other hand, emollient ingredient of jojoba oil is impregnated into pores of another porous micro powder of silica which is mixed with micro powders of polymethyl methacrylate. The emulsion encapsulated by CAB-O-SIL® powder and the jojoba oil impregnated porous micro silica powder are blended to form aggregates of oil impregnated micro porous powders surrounding an liposome encapsulated by powders of CAB-O-SIL®. Final product of aggregate is powder form in normal state. But, the powder changes to a liquid state when sprayed on a user's skin and apply pressure.

This application is a Continuation-in-Part of the previous application Ser. No. 10/122,360 which is now abandonded and the inventor is changed.

### FIELD OF THE INVENTION

Current application is related with a method of stabilizing high dose of cosmetic ingredients and a cosmetic product produced by the method thereof.

### BACKGROUND OF THE INVENTION

Active ingredients for multi-functional cosmetics are physically, and chemically unstable under normal atmospheric conditions and are easily decompose due to oxidation, and other chemical reactions. Such decomposition leads to discoloring and undesired odor. All the prior arts related with cosmetic products illustrate method of incorporating low dosage of the active ingredients into the cosmetic products to prevent such deterioration. A technique named ‘multi-encapsulation’ is developed and introduced to stabilize one of the active ingredients of cosmetic products by protecting it from deactivation. The technique enables maximizing of the functions of pure Vitamin C as a cosmetic active ingredient. It is the purpose of the current application to minimize the instability of the previously utilized function of active ingredient to provide improved functional cosmetic products those changes from powder state to liquid state when applied to a user's skin and slightly press the powder.

### DESCRIPTION OF THE PRIOR ARTS

U.S. Pat. No. 6,346,239 to Mallo, et al. illustrates a composition having an oil phase, an aqueous phase, at least one emulsifier of water-in-oil (W/O) type and at least one emulsifier of oil-in-water (O/W) type, in the form of a reverse latex comprising from 20% to 70% by weight, and preferably from 25% to 40% by weight, of an anionic polyelectrolyte, the anionic polyelectrolyte being based on partially neutralized acrylic acid, which may be branched and/or crosslinked, to prepare a cosmetic, dermopharmaceutical or pharmaceutical composition. The final product is in an aqueous form.

U.S. Pat. No. 6,048,546 to Sasaki, et al. illustratres a method for preparing encapsulated lipid-bilayer materials in a silica matrix comprising preparing a silica sol, mixing a lipid-bilayer material in the silica sol and allowing the mixture to gel to form the encapsulated lipid-bilayer material. They use gelation method of silica sol. The silica changed from solution to gelatin state. Only one ingredient is encapsulated by silica gel.

U.S. Pat. No. 6,020,367 to Duffy, et al. illustrates a method of preparing a supersaturated and stable solution of ascorbic acid, supersaturated ascorbic acid solutions and compositions containing such supersaturated solutions. A polyol vehicle is heated to an elevated temperature, and the ascorbic acid is dissolved therein to form an ascorbic acid/polyol solution. The final product is liquid state.

U.S. Pat. No. 5,993,851 to Foldvari illustrates a biphasic multilamellar lipid vesicle comprising a plurality of spaced apart lipid bilayers including a liposome forming component and optionally a biologically active agent entrapped within the lipid bilayers. The biphasic multilamellar lipid vesicle is in oily form.

U.S. Pat. No. 5,034,228 to Meybeck, et al. illustrates a composition comprising hydrous lipidic lamellar phases or liposomes containing, as an active agent a retinoid or a structural analogue of retinoid, such as a carotenoid or tretinoin.

U.S. Pat. No. 5,008,109 to Tin illustrates a process of stabilizing micellular particles such as vesicles and increasing the shelf life by suspending the particles in a polymeric gel matrix. The invention also relates to such particles suspended in the gel matrix with a protective gel surface thereabout which is capable of becoming fluid and converting the protective surface of an aqueous suspension.

None of the prior art illustrates method of preparing a powder form of vitamin C that changes into liquid form when spread on the user's skin and apply a slight pressure by a finger.

### SUMMARY OF THE INVENTION

With this disclosure, we report a, method called multi-encapsulation to stabilize rather unstable pure Vitamin C, which is highly desired ingredient in cosmetic industry. The brief description of the process is as followed. Powders of pure vitamin C are well blended with mixtures of glycerin and lecithin to form a liposome of liquid emulsion state. The emulsion, which is comprised of vitamin C, glycerin and lecithin, is encapsulated with micro powder of CAB-O-SIL® TS-530. On the other hand, emollient ingredient of jojoba oil is impregnated into other porous micro powder of silica having average particle size less than 1 micrometer that is mixed with micro-powders of poltmethylmethacrylate. The emulsion encapsulating CAB-O-SIL® powder and the mixture of micro-powders of poltmethylmethacrylate and jojoba oil impregnated porous micro silica powder are blended to form aggregates of mixture of micro-powders of poltmethylmethacrylate and jojoba oil impregnated porous micro silica powder powders surrounding an emulsion encapsulating CAB-O-SIL®. The final product is dried by freeze-drying technique to remove all the moist. The encapsulation is destroyed by physical pressure which is applied to the final product that contacts with a user's skin. Then the active ingredient, dissolved in solution phase, is transferred to the skin.

### BRIEF DESCRIPTION OF THE DRAWING

FIG. 1 is a schematic drawing of the procedure for forming aggregated products of the current application.

FIG. 2 is a schematic drawing showing the status change from a powder state to a liquid state of the product of current application when spread on a user's skin and applies a slight pressure.

### DETAILED DESCRIPTION OF THE PREFERRED EMBODIMENT OF THE INVENTION

The main purpose of this invention is to develop a new method to protect the unstable active ingredient, pure Vitamin C, by encapsulation to produce the stabilized powder. This blended with base formulation, would be dissolved and transferred to skin more effectively, maximizing the functional benefits of pure Vitamin C. Furthermore, the stabilized pure Vitamin C powder can be incorporated with high dosage to increase its activity.

The cosmetic products have attracted the attention of women of all nations not only due to their properties like protecting the skin from external factors like dryness, sun-light, etc, and preserving the purity of the skins, and keeping the skin moisture, preventing from being rough, but also their colors and fragrances, providing the women with more beauty.

However, the desire of the customers keeps evolving, hoping that the more special functions will be provided in the cosmetic products. Therefore, the cosmetic industry stated to focus their research on how to incorporate the active ingredients into their products. Albumin, lecithin, licorice, Vitamin C Vitamin C derivatives, retinol, tocopherol, salicylicacid, benzoyl peroxide, azelaic acid are the several example of the active ingredients that the cosmetic industries are interested in, and the most well-known methods for protecting the active ingredients is to make them as liposome. According to the reference C&T, 105, May, 1990, p65-78, C&T, 109, 1996, Fragrance/ J,1984, liposome method can temporally protect the active ingredients from the deactivating environment, so the active ingredients can have prolonged life time. Also the liposome is a good skin-penetrating agent, further increasing the efficiency of the active ingredients transfer. However, this liposome method alone could not stabilize all the active ingredients.

Even when the high dosage is incorporated, the effective activity of pure Vitamin C is lowered due to the fact that pure Vitamin C tends to undergo decomposition or oxidation by heat, light, moist and air, leading to discoloring and precipitation.

To come up with better solution, we invented a stabilization method called multi-encapsulation technique to wrap around the unstable pure Vitamin C with protecting capsule and blended with base formulation. This stabilized pure Vitamin C is kept as powder and when applied, the pure Vitamin C is dissolved into skin safely. This is a highly improved technique compared to the precedent methods, since the pure Vitamin C can be applied with high concentration so that its activity

### Experiments

1. Preparation of the Product

1-1 Materials and Contents

The product before the Freeze-drying is comprised of 1.0˜30 wt % of porous powder, 0.1˜20 wt % of liposome, 7.1˜15 wt % of CAB-O-SIL®, 60˜90 wt % of solvent, and 0.1˜15 wt % of the active ingredient, vitamin C. The porous powder used in this procedure is polymethyl-methacrylate and silica. Using the porous powder less than 1.0 wt % resulted in decrease in the degree of encapsulation of liposome and loss of stability. Using the porous powder more than 30 wt % resulted in uncomfortable dryness due to the strong adsorption power of porous powder. Sunsil®-130, Silica Microbead L-15000, Microsphere BPA-500, and Micropearl M-101 are used for the source of polymethyl-methacrylate and silica.

Using the liposome more than 20 wt %, the liposome is not easily encapsulated by the porous powder. The main component of the liposome is one and/or mixture of squallene, glycerol trioctanoate, capril carprilic triglyceride, cyclomethicone, jojoba oil, tocopherol acetate, lecithin, polyglyceryl myristate, glycerin, stearic acid, cetanol, phytosphigosine, polyglyceril methacrylate, sodium hyaluronate, distilled water, etc were mixed together. This liposome provides initial protection. Using the CAB-O-SIL® less than 7.1 wt % results in unstable solution phase, and increase the chance of coagulation by physical shock. Using the CAB-O-SIL® more than 15 wt % forms concentrated solution that gives dry feeling and leaves nebula on the skin.

For the solvents, we used water and one or the mixture of two from the following commercially available chemicals such as glycerin, diglycerin, triglycerin, proply glycerin, 1,3-butyl glycol, hexylene glycol, sorbitol, pentenol, sodium hyaluronate, trehalose. Using solvnt less than 60 wt % causes solubility issue, and usng more than 90 wt % forms less stable powder.

Pure Vitamin C is used as active ingredient. Other ingredients such as albumin, lecithin, glycyrrhizin acid, retinol, retinol palmitate, vitamin E Vitamin E acetate, salicylic acid, benzoyl peroxide, and azelaic acid are considered as an active ingredient. The amounts of those active ingredients is determined by HPLC.

1-2 Preparation of the Powder Product;

The detailed procedure of making our encapsulation product is as follows;

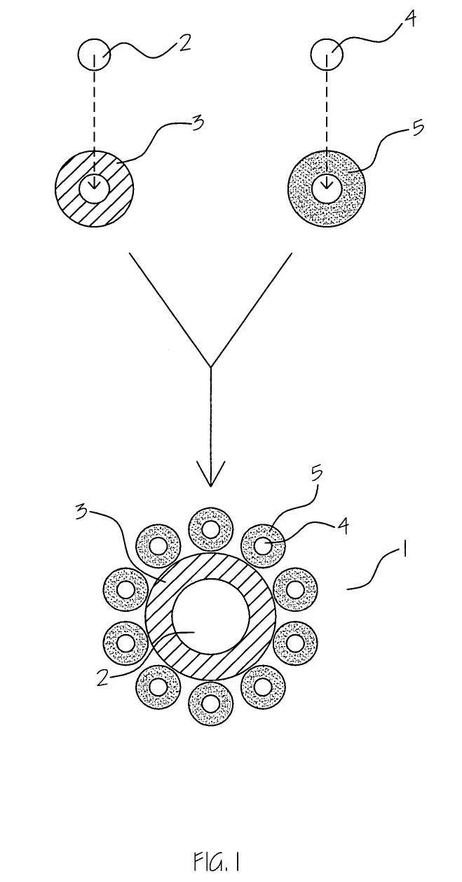

FIG. 1 is a schematic drawing of the procedure for forming aggregated products (1) of the current application.

Vitamin C is prepared as a liposome by dissolving it into liposome ingredient such as squalene, glycerol trioctanoate and distilled water, etc. Encapsulate the vitamin C containing liposome (2) with micro silica powder, CAB-O-SIL® TS-530 (3) by mixing them homogeneously with an ultra-fast mixer.

Emollients (4) are pregnated into the pores of another porous micro silica powder (5) by adding emollient little by little to the bed of the porous powder. Mix them homogeneously with the ultra-fast mixer to make powder form.

The encapsulated vitamin C containing liposome is encapsulated again with emollient pregnated porous silica. Residual water is removed by Freeze drying method to produce a multi-encapsulate powder containing pure Vitamin C that gives a special function to cosmetic product.

2. Stability of the Final Product;

Stability of the product was measured by the following experiments;
i) Stability towards physical contact:
J. Engelsmann's volumeter, which is frequently used to test the stability of the powder, was used to test the stability of the product towards physical shock. The device provides impulse to the powder by vibrating the powder up and down, and measuring the degree of coagulation tells the degree of stabilization.
Followings are the detailed procedure:
   40 grams of the powder was added to a 100 ml flask, and vibrated 100 times.
   The above powder was filtered with filter funnel size 50 (300 micrometer).
   The mass of the substance left on the filter, which is the destroyed capsule, was measure and the weight percentage was calculated.
ii) Moistening effect:
The moistening effect was measured by a group of selected people who would testify the moistening effect of the products of the current application.
iii) Heat stability of the product:
Heat stability of the powder of the current application was estimated by measuring the amount left after six months at 25° C. and three months at 40° C. with HPLC (High Pressure Liquid Chromatogram).
iv) Effect of Freeze-drying on the stability of the product:
To improve the stability, Freeze-drying method is applied to get rid of the moist.

The below shows the detailed experiments, and tests.

Several samples with different chemical composition were synthesized ultra-fast mixer as shown in Table 1. 7 samples were prepared. The first three whose composition ratio of the chemicals were outside the require ratio, were labeled as comparison 1, 2, and 3, and the last four with correct compositions were labeled as Good sample 1, 2, 3, and 4. Then we have tested these samples on their fineness, moisturizing, and their degree of purity as a function of time. Following are the procedures and the grades for those samples prepared.

2-2 Procedure for the Tests, and their Grades.
I) The fineness: 20 grams of the samples were filtered through the filter funnel with size 50 (300 um), and the filtrate was weighed again.

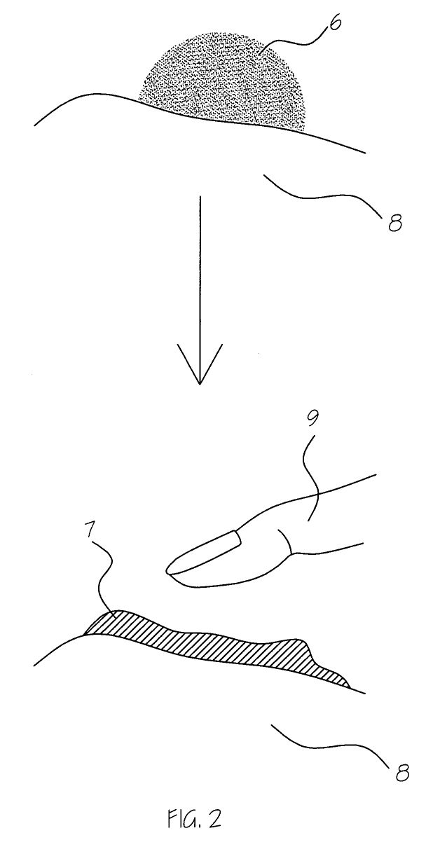

A; 95% to more passed through the filter, B;90 to 95% passed through the filter, C;85 to 90% passed through the filter, D;80 to 85% passed through the filter, E; 75 to 80% passed through the filter, F; 75% or less passed through the filter.
II) Moisturizing activity: FIG. 2 is a schematic drawing showing the status change from a powder state (6) to a liquid state (7) of the product of current application when spread on a user's skin (8) and applies a slight pressure with a finger (9).

When a user spread the sample to the skin (8) of a user and presses the samples, the powder surprisingly changes to liquid. The samples were prepared and applied to the skin and tested how moisture the samples were A: Very moisture, B: Moisture, C: Average, D: Dry, E: Very Dry
III) The degree of purity over the time: To determine the degree of purity over the time, the sample were left at 25 C for six months and then injected to HPLC to measure how many pure samples were left after 6 months.
   A: The purity of 95% or higher, B: The purity of 90-95%, C: The purity of 85-90% D: The purity of 80-85%, E: The purity of 75-80%, F: The purity of 75% or below
IV) The stability towards the physical shock: We used J. Angel's man (?)'s scam volume meter to test the stability of the product towards physical shock.

40 grams of the sample were placed in 100 ml flask, and vibrated 100 times. The above powder was filtered with filter funnel size 50 (300 um). The mass of the substances left on the filter which is the destroyed capsules were measured and the weight percentage was calculated.

A: 5% or less destroyed, B: 5˜10% destroyed, C: 10˜15% destroyed, D: 15˜20% destroyed, E: 20% or more destroyed.

2-3. The Results of the Tests

The result of the testing 7 samples showed good stability of the encapsulated pure Vitamin C against physical shock, over the time, and high degree of fineness and good moisturizing activity. Those three Comparison samples that had the incorrect chemical composition were graded below average for the fineness of the power and purity over the time, meaning the stability of encapsulated pure Vitamin C was low. However, four Good samples showed improvements for all of the categories of the tests, demonstrating the power of the multi-encapsulation technology.

TABLE 1

| | | Comparison | | | Good Samples | | | |
|---|---|---|---|---|---|---|---|---|
| Chemical | | 1 | 2 | 3 | 1 | 2 | 3 | 4 |
| Silica Silate | | 3.0 | 5.0 | 20.0 | 7.1 | 10 | 15 | 8.5 |
| Glycerin | | 20 | 20 | 20 | 40 | 50 | 20 | 58.7 |
| 1,3-butylene glycol | | 15 | 15 | 15 | 5 | 10 | 15 | 5 |
| Pentenol | | 0.5 | 0.5 | 0.5 | 0.5 | 0.5 | 0.5 | 0.5 |
| Paraben | | 0.3 | 0.3 | 0.3 | 0.3 | 0.3 | 0.3 | 0.3 |
| Distilled Water | | The rest | The rest | The rest | Rest | Rest | Rest | Rest |
| Liposome | | 0.05 | 25 | — | 0.3 | 4.0 | 8 | 5 |
| Porous Powder | | 0.1 | 0.5 | 0.8 | 1.0 | 15 | 20 | 12 |
| Pure Vitamin C | | 0.01 | 0.05 | 20 | 5 | 10 | 15 | 10 |
| Grading | Fineness | E | D | A | A | A | A | A |
| | Physical shock | E | D | A | A | A | A | A |
| | Moisture | D | A | E | B | B | C | A |
| | Purity over time | C | C | C | C | B | B | A |

The followings are the conclusion drawn from the testing. The idea to block the interaction between the water and the active ingredient, pure Vitamin C by covering it by liposome capsules powder, indeed worked well, and also removing water which resided in thecapsules by Freeze-dry technique, prolonged the life-time of the pure Vitamin C.

From the results of the testing, we developed following four products.

1. Powder with Whitening Activity.

| | |
|---|---|
| Silica Silate | 8.0 wt % |
| Glycerin | 60.0 wt % |
| Distilled water | the rest |
| 1,3-butylene glycol | 5.0 wt % |
| paraben | 0.3 wt % |
| pentenol | 0.5 wt % |
| liposome | 6.0 wt % |
| porous powder | 10.0 wt % |
| Pure Vitamin C | 10.0 wt % |

The above product contains 10% of pure Vitamin C which has superior whitening effect. Since the pure Vitamin C is stabilized as the capsules, the life time of the pure Vitamin C is very long. Even when heated at 40 C for 3 months, the purity of the active ingredient was higher than 90% ( determined by HPLC)

2. Anti-Wrinkle Powder

| | |
|---|---|
| Silica Silate | 10.0 wt % |
| Glycerin | 63.0 wt % |
| Distilled water | the rest |
| 1,3-butylene glycol | 5.0 wt % |
| paraben | 0.3 wt % |
| pentenol | 1.0 wt % |
| liposome | 8.0 wt % |
| porous powder | 12.0 wt % |
| Pure Retinol (1,420,000 IU/g) | 0.2 wt % |

The above product contains Retinol (3,000 IU) which has anti-wrinkle activity.

3. Acne Powder

| | |
|---|---|
| Silica Silate | 8.0 wt % |
| Glycerin | 20.0 wt % |
| Distilled water | the rest |
| 1,3-butylene glycol | 10.0 wt % |
| paraben | 0.3 wt % |
| pentenol | 0.1 wt % |
| liposome | 8.0 wt % |
| porous powder | 12.0 wt % |
| Salicylic Acid | 0.5 wt % |

The above product contains salicylic acid which prevents acne.

4. Keratin-Removing Powder

| | |
|---|---|
| Silica Silate | 8.5 wt % |
| Glycerin | 40.0 wt % |
| Distilled water | the rest |
| paraben | 0.3 wt % |
| pentenol | 0.5 wt % |
| lipsome | 8.0 wt % |
| porous powder | 12.0 wt % |
| AHA | 0.5 wt % |

The above product contains high concentration of AHA which can remove keratin. Since AHA is stabilized in the capsules, the life time of AHA is very long. Even when heated at 40 C for 3 months, the purity of the active ingredient was higher then 90% (determined by HPLC).

As stated above, the new technology to encapsulate the active ingredients to stabilize them from decomposition and keep them as fine powder was developed. The cosmetic product were made from this stabilized active ingredients blended with the base formulation. These active ingredients in the capsules were not destroyed by physical shocks and penetrated well into skin, resulting maximum anti-wrinkle, whitening, acne, and keratin-removing effect. Not only has this method provided the stability of the active ingredients, but also the ease for use, and excellent moistening effect.

1. A powder form aggregate, which is changed to liquid form when sprayed on a user's skin and a slight pressure is applied, is comprised of 1) a liposome containing cosmetic ingredient, solvent and liposome agent, which is encapsulated with micro particle silica treated with hexamethyldisilazane, CAB-O-SIL® TS-530, 2) porous powders, having average particle size less than 1 micrometer, impregnated with emollient ingredient of jojoba oil within their pores and encapsulating the CAB-O-SIL® encapsulated liposome.

2. A powder form aggregate of claim 1, wherein the cosmetic ingredient is vitamin C.

3. A powder form aggregate of claim 1, wherein the final powder form of aggregate is prepared by Freeze-drying method.

4. A powder form aggregate of claim 1, wherein the composition and Components are 15 wt % of porous powder, 10 wt % of liposome, 10 wt % of CAB-O-SIL®TS-530, 75wt % of solvent and 15 wt % of pure Vitamin C.

5. A powder form aggregate of claim 1, wherein the porous powder is mixture polymethyl-metharylate and silica.

6. A powder form aggregate of claim 1, wherein the liposome is comprised of mixture of squallene, glycerol trioctanoate, capril caprilic triglyceride, cyclomethicone, jojoba oil, tecopherol acetate, lecithin, polyglyceryl myristate, glycerin, stearic acid, cetanol, phytosphigosine, polyglyceril methacrylate, sodium hyluronate, and distilled water.

7. A powder form aggregate of claim 1, wherein the solvents is comprised of glycerin, diglycerin, triglycerin, proply glycerin, 1,3-butyl glycol, hexylene glycol, sorbitol, pentenol, sodium hyaluronate, trehalose, and distilled water.

8. A powder form aggregate of claim 1, the active ingredient is pure Vitamin C.

9. A powder form aggregate of claim 1, wherein the active ingredient is albumin.
